(19) United States
(12) Patent Application Publication
(10) Pub. No.: US 2002/0087129 A1
(43) Pub. Date: Jul. 4, 2002

(54) Absorbent article having releasable medicinal treatments

(75) Inventors: Robert Cosmo Di Luccio, Titusville, FL (US); Alice Susan Gordon, Roswell, GA (US); Duane Gerard Krzysik, Appleton, WI (US); Varunesh Sharma, Atlanta, GA (US)

Correspondence Address: William W. Letson, Kimberly-Clark Worldwide, Inc., Patent Department, 401 North Lake Street, Neenah, WI, 54956, US

(73) Assignee: Kimberly-Clark Worldwide, Inc.

(21) Appl. No.: 09/990,697

(22) Filed: Nov. 16, 2001

Related U.S. Application Data
(60) Provisional application No. 60/259,143, filed on Dec. 29, 2000.

(51) Int. Cl. A61M 35/00; A61F 13/00; A61F 13/15; A61F 13/20

(52) U.S. Cl. 604/304; 604/289; 604/359; 604/360; 604/364; 604/385.06

(57) ABSTRACT

The present invention relates to articles, such as a personal care product, having (i) a topsheet having a body-facing surface, said topsheet having a medicinal composition pattern applied to a region of the body-facing surface of the topsheet in an effective amount and wherein said pattern comprises individual segments having at least one dimension less than about 4 millimeters (extending in the MD and CD plane); and (ii) an absorbent material positioned adjacent the topsheet. In a further aspect, the article may further include a backsheet, which is desirably liquid-impervious, and which is positioned adjacent the absorbent material and opposite the topsheet. The present invention is also directed to a process for making said absorbent articles.

### BACKGROUND OF THE INVENTION

Absorbent articles, such as infant diapers, adult incontinence garments, sanitary napkins, bedpads, panty liners, incontinent pads, feminine hygiene products, and so forth are well known in the art. These articles are inexpensive, often disposable, and yet capable of absorbing and retaining fluids and other bodily exudates. The most basic design of all such articles typically includes a bodyside layer or topsheet, an outercover or backsheet and an absorbent core disposed between the bodyside layer and the backsheet; however, it is of note that not all such articles have the outercover or backsheet (e.g. some feminine hygiene products such as a tampon). In those instances where a backsheet is not present, the outercover or topsheet generally encases the absorbent core. Generally, the bodyside liner and the outercover are sealed about the periphery so as to encapsulate the absorbent core and thus make it possible to entrap and retain any fluids contained within the absorbent core. Depending upon the design of the particular personal care absorbent article, other components also may be included. Thus, the product may include such things as elastic side panels, fluid containment flaps, fastening devices and other layers of fluid transfer or retention materials.

Various topsheets are known in the art and they are commonly designed to provide good liquid handling properties and promote the flow of body exudates away from the skin and towards the absorbent core. However, with certain products, extended wear of a soiled article can cause the skin to be exposed to extremely high humidity and therefore cause wearers to experience some level of skin irritation including, but not limited to, erythema, diaper rash and loss of skin barrier. The same is true of those products which are used internally (e.g. tampons) or which are exposed to wounds and the like, except that the surface of the body to which those products are exposed may have different causes of irritation (e.g. frictional damage, over drying (tampons or interlabial devices can absorb natural moisture)). In addition, with regard to diapers and similar personal care products, while they efficiently take in and absorb urine, they do not efficiently absorb fecal materials and fecal solids or adequately remove fecal materials and fecal solids away from the skin. The same can generally be said of feminine hygiene products in that the fluid of the menses is taken into or absorbed into the product, but the solids are not typically absorbed or moved away from the surface of the skin or outermost body surface to which the products are exposed.

In order to address these issues, it is a common practice for caregivers to apply a lotion, cream and/or other medicament to the wearer's skin prior to putting the absorbent article on or adjacent the individual. As a particular example, it has been common for a caregiver to apply Vaseline or mineral oil to those external areas covered by the diaper prior to putting the diaper on the infant. This helps the skin by acting as a lubricant, acting to improve or provide skin barrier as well as help prevent irritants from adhering to the wearer's skin. However, in an effort to avoid the need for manual application of skin protectants and/or other medicaments, emollients have been applied to the skin via the absorbent article itself. As an example, US Patent No. 3,585,998 teaches a disposable diaper having an interior layer with capsules of emollients that rupture under pressure. The emollient is then transferred to the skin by normal contact, wearer motion and/or body heat. As a further example, U.S. Pat. No. 5,643,588 teaches coating a semi-solid emollient on the surface of a topsheet in a personal care article such that the emollient is likewise transferred to the wearer's skin by normal contact, wearer motion and/or body heat.

As a particular example, the PAMPERS Rash Guard diaper utilizes a topsheet containing emollients located on the body-side of the sheet and arranged on the topsheet in a striped pattern, where the stripes run in the article's longitudinal direction. Typically, stripes are applied wherein each stripe measures about 0.25 inches wide (i.e., in the article's lateral direction) and 11.75 inches long. The distance between the stripes is approximately 0.3 inches. Application of skin care compositions to diaper topsheets are further described in WO 99/45973.

One problem with application of emollients to the topsheet is that they often interfere with the intended function of the topsheet, namely to quickly and efficiently take in and distribute liquids through the topsheet, away from the wearer, and to the absorbent core. The emollients as applied to the topsheet often form films or otherwise obstruct the pores due to their structural and hydrophobic properties and therefore application of emollients to topsheets often degrades the liquid handling properties of the topsheet. Thus, the benefits that can be obtained by applying emollients to the topsheet often compete with the liquid distribution and liquid-handling properties sought to be obtained by the topsheet. Accordingly, it is often necessary to limit the amount of emollient applied to the topsheet. However, in order to provide the desired therapeutic effect to the skin, adequate emollient needs to be available for and capable of release and transfer to the wearer's skin.

An additional problem with existing absorbent articles and processes is that the application of the emollients to the topsheet is uneven in terms of location and/or amount. Such an uneven application of emollient often results in a corresponding uneven transfer of the emollient to the skin. This is disadvantageous in that some areas of the skin may receive little or no emollient whereas other areas may receive excess amounts of emollient. Thus, the skin may have numerous areas that, by reason of not receiving an effective amount of emollient or by receiving an excessive amount of emollient, experience one or more disadvantages. Further, the inability to uniformly apply the emollient also makes it difficult to efficiently utilize the emollients applied to the topsheet.

Therefore, absorbent articles are desired having body-side layers or topsheets with greater amounts of medicaments available for release without significantly detracting from the functional properties of the topsheet. Further, absorbent articles are desired having body-side layers or topsheets capable of providing significant release and transfer of medicaments to the wearer's skin. Still further, such body-side layers and/or topsheets are desired having a uniform application of medicaments thereon.

### SUMMARY OF THE INVENTION

The present invention relates to articles having (i) a topsheet having a body-facing surface, said topsheet having a medicinal composition pattern applied to a region of the body-facing surface of the topsheet in an effective amount and wherein said pattern comprises individual segments having at least one dimension less than about 4 millimeters (extending in the MD and CD plane); and (ii) an absorbent material positioned adjacent the topsheet. In a further aspect, the article may further include a backsheet, which is desirably liquid-impervious, and which is positioned adjacent the absorbent material opposite the topsheet. In one aspect of the present invention, a micro-dispense valve jet, will be used to apply the medicament or medicament composition and the patterned application of the medicinal composition may include a plurality of substantially continuous lines having a desired width in the range of about 1.5 millimeters to about 3 millimeters, and most desirably in the range of about 1.5 to about 2.5 millimeters. In a further aspect, the patterned application of the medicinal composition may be applied by a piezoelectric jet and may include a plurality of substantially continuous lines having a width less than about 1 millimeter and desirably less than about 0.9 millimeters, and even more desirably having a width between about 0.9 and about 0.05 millimeters. Still further, the medicinal composition may be present upon said body-facing surface of said topsheet in an amount between about 0.05 mg/cm² and about 50 mg/cm². In yet a further aspect, the patterned medicinal composition is desirably substantially uniformly applied to the selected regions. In yet another particular aspect, the pattern can comprise a matrix of discrete segments and further the discrete segments can have a length and width less than about 4 mm. In yet still another particular embodiment, the pattern can comprise a matrix of discrete segments and further the discrete segments can have a length and width less than about 0.9 mm. In a further aspect, the discrete segments can comprise round or dot-like segments and also have a substantially rounded cross-sectional shape. In yet a further aspect, the discrete segments of medicinal composition may each have a volume of between about 3 picoliters and about 400 nanoliters.

In a further aspect of the invention, an absorbent article is provided comprising (i) a topsheet having a body-facing surface, said topsheet comprising a porous material and having a medicinal composition upon the body-facing surface of the topsheet in an effective amount and wherein said pattern comprises a matrix of discrete segments having a volume of between about 3 picoliters and about 400 nanoliters; and (ii) an absorbent material positioned adjacent the topsheet. In a further aspect, the article may further include a backsheet, which is desirably liquid-impervious, and which is positioned adjacent the absorbent material opposite the topsheet. The discrete segments may have a substantially semicircular cross-section extending above the body-facing surface of the topsheet. Further, the discrete segments may comprise substantially round segments (in the MD and CD plane). Still further, the discrete segments are desirably positioned upon said topsheet in a frequency of between about 1 Hz to about 100 kHz. Generally, the frequency of segment positioning may be described in terms of the number of times an applicator “fires” or pulses in a given time so as to produce the segments. Still further, the discrete segments of medicinal composition may be limited to selected regions of the topsheet. Further, the medicinal composition can be applied to the selected regions in a series of spaced lines extending across the body-facing surface of the topsheet.

The present invention is also directed to a process for producing a medicament treated material, the process including providing a substrate; and applying a medicament composition to the substrate in discrete segments; wherein the medicament composition is present on the substrate in a frequency of about 1 Hz to about 100 kHz. One of skill in the art will recognize that some of the application methods described in more detail below will work better than others within the ranges specified herein and that the applicator should be selected accordingly. The step of providing a substrate may include the provision of a woven, nonwoven, film or laminate thereof. The substrate may produced in-line or off-line. It is contemplated that the discrete segments may be located upon selected regions of the substrate. The medicament of the present invention may be applied to the substrate in a pattern, which may be random or repeating. While any suitable medicament chemistry is contemplated by the present invention, the medicaments in this invention can include but are not limited to lipid chemistries (barrier repair and protection), skin protectants actives, antimicrobials actives, antifungals actives, astringent actives, deodorants, external analgesics actives, sunscreen actives and the like. Desirably, the discrete placement of the medicament may be achieved by micro-dispense valve (or “valve jet”), a piezoelectric jet (or “piezo jet”), spraying or any non-contact means of application.

If the medicament is applied via piezo jet, it is desired that the viscosity of the medicament applied to the substrate be up to about 25 centipoise, more desirably about 4 centipoise to about 20 centipoise, and most desirably about 8 centipoise to about 14 centipoise, at the time of application. Furthermore, it is desired that the pattern of medicament include a matrix of discrete segments having a volume of between about 3 and about 200 picoliters each. Further still, it is even more desired that the discrete segments may be placed upon substrate in a frequency between about 10 kHz to about 50 kHz, and more desirably at a frequency of up to about 40 kHz, wherein the frequency is a measure of the number of times an applicator “fires” or pulses in a given time so as to produce the segments. It is of note that the frequency expressed in Hz is not the number of times the applicator must fire to produce one discrete segment, but rather is the number of times the applicator may fire in a given time period so as to produce a number of discrete segments over the desired region or regions of the substrate. Furthermore, one skilled in the art will recognize that the speed or frequency at which something, (e.g. a medicament) may be applied to a substrate does not describe the positioning of the resulting discrete segments relative to one another. That is, without an indication as to the velocity of the substrate to which the segments are being applied, the segments could be applied so as to be on top of each other or have a great spacing therebetween.

Accordingly, while only the frequency of the applicators is discussed in detail herein, one skilled in the art will recognize that the substrate velocity may be selected and/or controlled so as to produce the desired segment position interval or intervals.

If the medicament is applied via valve jet, it is desired that the process viscosity of the medicament applied to the substrate in a range of about 1 centipoise to about 300 centipoise, more desirably about 4 centipoise to about 50 centipoise at the time of application. Furthermore, it is desired that the pattern of medicament include a matrix of discrete segments having a volume of between about 5 and about 400 nanoliters. Further still, it is desired that the discrete segments may be placed upon substrate in a frequency between about 1 Hz to about 2 kHz, and more desirably at a frequency of about 500 Hz to about 1.2 kHz.

### DESCRIPTION OF THE INVENTION

Definitions

As used herein and in the claims, the term “comprising” is inclusive or open-ended and does not exclude additional unrecited elements, compositional components, or method steps. Accordingly, the term “comprising” encompasses the more restrictive terms “consisting essentially of” and “consisting of.”

As used herein, all percentages, ratios and proportions are by weight unless otherwise specified.

As used herein, the term “body-side” or “inner-side” refers to the side of a material that will face the wearer of the article and the term “outer-side” refers to the opposing side that faces away from the body, i.e. distal to the body when the article incorporating the material is worn.

As used herein, the term “effective amount” means an amount capable of providing a therapeutic benefit to the skin and takes into account the amount of medicament or medicament composition needed to be delivered to the skin in order to achieve the benefit as well as the percent of medicament normally released from the substrate and delivered to the skin.

As used herein, the term “fabric” means a material comprising a network of fibers including, but not limited to, woven or knitted materials, tufted or tufted-like materials, nonwoven webs, and so forth.

As used herein, the term “machine-direction” or MD means the direction of a fabric in the direction in which it is produced. The term “cross-direction” or CD means the direction of a fabric generally perpendicular to the MD.

As used herein, the term “medicament” refers to any compound or composition that provides a benefit or therapeutic effect upon and/or to the skin by physical contact with the skin. This benefit or therapeutic effect can be achieved upon initial application and/or over time with continued use. As used herein the terms “medicament” and “medicament composition” may be used interchangeably.

As used herein, the term “nonwoven” fabric or web means a web having a structure of individual fibers or threads which are interlaid, but not in an identifiable manner as in a knitted or woven fabric. Nonwoven fabrics or webs have been formed by many processes such as, for example, meltblowing processes, spunbonding processes, hydroentangling, airlaid and bonded carded web processes.

As used herein, the term “porous” refers to a substrate or material that has interstitial spaces or openings located therein such that there exist pathways that extend through the entire thickness of the material, the interstitial spaces need not extend through the entirety of the material and can collectively form pathways through the thickness of the material via adjacent, inter-connecting spaces.

As used herein, the term “personal care product” means personal hygiene oriented items such as wipes, diapers, training pants, absorbent underpants, adult incontinence products, feminine hygiene products, and so forth. Personal care products also include nursing pads, wound care articles, pads or bandages, mortuary and veterinary products and wipes, time and/or condition release articles (such as medicine patches, etc.) and the like.

### BRIEF DESCRIPTION OF THE DRAWINGS

FIG. 1 is a drawing of an absorbent article of the present invention having a topsheet with a medicinal treatment thereon and wherein a portion of the body-facing surface of the topsheet is depicted in an enlarged view.

FIG. 2 is a cross-sectional view of a topsheet having a medicament composition thereon.

FIG. 3 is a schematic representation illustrating an exemplary method for applying a medicinal treatment to a porous sheet.

FIGS. 4a and 4b are cross-sectional views of one embodiment of a piezoelectric jet. FIG. 4a shows the jet in its rest position. FIG. 4b shows the jet in a pulse position.

FIG. 5 is a cross-sectional embodiment of an article of the present invention, wherein the article does not include a backsheet.

### ABSORBENT ARTICLES

Absorbent articles generally include a liquid permeable topsheet, which faces the wearer, and a backsheet or outer cover. Disposed between the topsheet and outer cover is an absorbent core, often the topsheet and outer cover are sealed to encase the absorbent core. Although the following detailed description will be made in the context of a disposable diaper, one skilled in the art will appreciate that the concepts of the present invention would also be suitable for use in connection with other types of absorbent articles, particularly other personal care products. In addition, although the present invention is described in the context of several specific configurations, it will also be appreciated that further combinations or alterations of the specific configurations discussed below may be made by one skilled in the art without departing from the spirit and scope of the present invention, including, but not limited to tampons and the like, which do not include a backsheet.

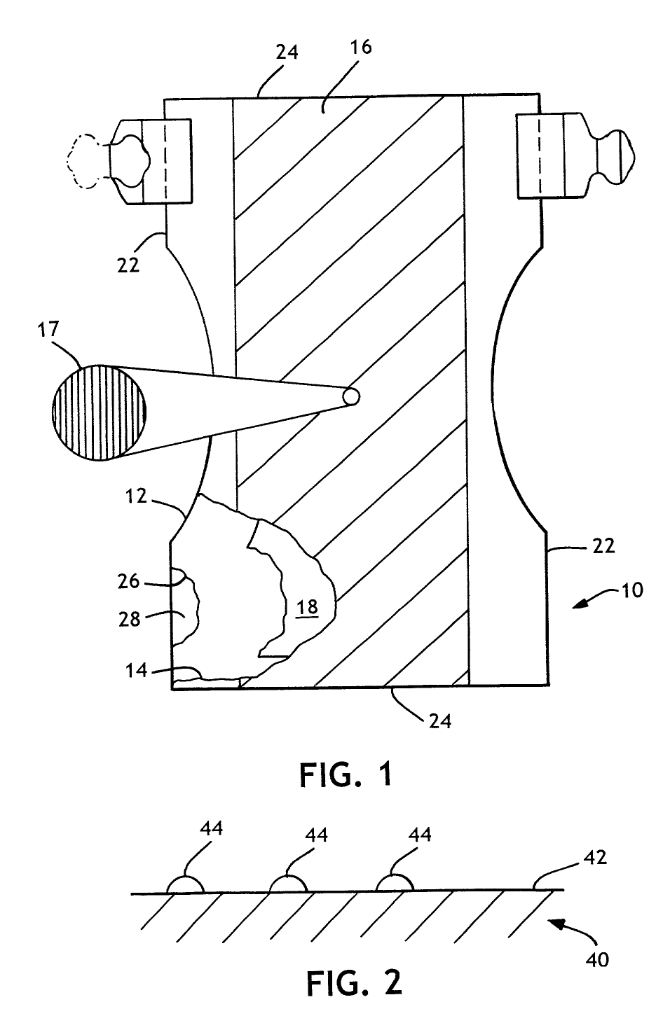

In reference to FIG. 1, diaper 10 may comprise a backsheet or outer cover 12, a topsheet 14, which is desirably liquid permeable, positioned in facing relation to outer cover 12, and an absorbent core 18 positioned between outer cover 12 and topsheet 14. Diaper 10 may be of various shapes as desired such as, for example, an overall rectangular shape, T-shape or an hourglass shape. Topsheet 14 is generally coextensive with the outer cover 12 but may optionally cover an area that is larger or smaller than the area of outer cover 12, as desired. Portions of diaper 10, such as a marginal section of the outer cover 12, may extend past the terminal edges of the absorbent core 18. In the illustrated embodiment, for example, outer cover 12 can extend outwardly beyond the terminal marginal edges of the absorbent core 18 to form side margins 22 and end margins 24 of the diaper 10. A treated region 16 containing medicinal treatment 17 is located upon a selected portion of the body-facing side of topsheet 14.

Topsheet 14 desirably presents a body-facing surface that is compliant, soft to the touch, and non-irritating to the wearer's skin. Topsheet 14 should have good liquid distribution and handling properties and, desirably, is also capable of isolating the wearer's skin from liquids held in the absorbent core 18. In order to present a drier surface to the wearer, the topsheet 14 may be less hydrophilic than the absorbent core 18 and also sufficiently porous to be readily liquid permeable. Various topsheets are well known in the art and may be manufactured from a wide variety of materials, such as porous foams, reticulated foams, apertured plastic films, natural fiber fabrics (e.g., wool or cotton fibers), synthetic fiber fabrics (e.g., polyester, polypropylene, polyethylene, etc.), or fabrics comprising a combination of natural and synthetic fibers. Desirably the topsheet comprises a highly open or porous fabric having numerous interstitial spaces therein. For example, the topsheet may comprise a high-loft spunbonded web of polyolefin fibers or a bonded-carded web of thermoplastic polymer fibers. In this regard, the topsheet may be composed of substantially hydrophobic material treated with a surfactant or otherwise processed to impart the desired level of wettability and/or permeability. Exemplary topsheets include, but are not limited to, those described in U.S. Pat. No. 5,879,343; U.S. Pat. No. 5,490,846; U.S. Pat. No. 5,364,382 and commonly assigned U.S. patent application Ser. No. 09/209,177 filed Dec. 9, 1998 (and related case WO 00/33779, published on Jun. 15, 2000); the entire contents of each of the aforesaid patents and applications are incorporated herein by reference. As a particular example, an exemplary liner may comprise two or more layers wherein the body-facing layer comprises a low-density nonwoven web of hydrophobic fibers and the underlying layer comprises a low-density nonwoven web of hydrophilic fibers.

Backsheet or outer cover 12 desirably comprises a liquid-impervious material and, even more desirably, comprises a breathable liquid-impervious material. The outer cover can itself comprise a single layer or a multilayer structure, e.g. a multilayer laminate. In reference to the particular embodiment depicted in FIG. 1, outer cover 12 may comprises a breathable liquid-impervious film 26 and one or more additional fabric layers 28. The particular structure and composition of the outer cover may be selected from various combinations of films and/or fabrics. The individual layers or laminates can be selected to provide a material having the desired attributes such as, for example, strength, abrasion resistance, water vapor transmission rate, tactile properties and/or aesthetics. Exemplary materials suitable for use as outer covers or as a component of an outer cover include, but are not limited to, those described in U.S. Pat. No. 5,213,881; U.S. Pat. No. 4,777,073; U.S. Pat. No. 5,855,999 and U.S. Pat. No. 6,075,179; the entire contents of each of the aforesaid patents are incorporated herein by reference. As a particular example, an exemplary outer cover material can comprise a breathable film laminate having a microporous polyolefin film laminated to a nonwoven web of polyolefin spunbond fibers.

Between outer cover 12 and topsheet 14 is positioned an absorbent core 18 which includes an absorbent material. Suitable absorbent materials include, but are not limited to, superabsorbent particles, wood pulp fluff fibers, synthetic wood pulp fibers, synthetic fibers and combinations thereof. Exemplary superabsorbent particles include, but are not limited to, silica gels, polyacrylamides, polyvinyl alcohol, ethylene maleic anhydride copolymers, polyvinyl ethers, hydroxypropyl cellulose, acrylonitrile grafted starch, acrylic acid grafted starch, polyacrylates, and mixtures or combinations thereof. Superabsorbent particles are commonly used in conjunction with other absorbent materials such as, for example, pulp fluff. In this regard, a mixture of pulp fluff and superabsorbent particles is commonly employed as an absorbent core in personal care articles such as diapers and incontinence garments. Superabsorbent materials may be substantially homogeneously mixed with other hydrophilic fibers or may be selectively placed into desired zones of the absorbent body to better contain and absorb body exudates. The concentration of the superabsorbent materials may also vary through the thickness of the absorbent core. Alternatively, the absorbent core may comprise a laminate of fibrous webs and superabsorbent materials or other suitable means for maintaining superabsorbent in localized areas. Additionally, when utilizing pulp or other like absorbents it is often advantageous to add a stiffer reinforcing fabric or material to the absorbent core in order to maintain the integrity of the absorbent after taking in liquid. The absorbent core may have any of a number of shapes. For example, the absorbent core may be rectangular, I-shaped, or T-shaped. It is generally desired that the absorbent core be narrower in the crotch area than in the front or rear portions of the diaper. The size of the absorbent core and selection of materials therein will vary with the desired loading capacity, the intended use of the absorbent article and other factors known to those skilled in the art.

While the basic structure of a diaper has been discussed hereinabove, it will be understood by those skilled in the art that numerous other components and/or structures may be utilized in the absorbent articles and personal care articles of the present invention. Notably, with regard to diapers, it will be readily appreciated that the diaper could include additional components such as for example elasticized side-panels, fasteners and/or elasticized leg cuffs that help secure the diaper to the wearer and reduce leakage from the diaper. As a further example, the diaper can include longitudinally extending containment flaps which are configured to maintain a substantially upright, perpendicular arrangement along the central portion of the diaper to serve as an additional barrier to the lateral flow of body exudates. These and other components are well known and the manner and method of using the same in connection with the absorbent article of the present invention will likewise be readily appreciated by those skilled in the art. By way of example only, exemplary personal care articles and components thereof are described in U.S. Pat. No. 4,798,603, U.S. Pat. No. 4,753,649, U.S. Pat. No. 4,704,116 and U.S. Pat. No. 5,429,629; the entire contents of each of the aforesaid patents are incorporated herein by reference.

### Medicaments

Numerous medicaments are known that are capable of providing therapeutic or health benefits to the skin. As used herein, medicaments include, but are not limited to, those compositions and materials capable of softening, soothing, placticize, moisturizing, coating protecting, (e.g. preventing or inhibiting irritants from adhering to the skin or otherwise degrading the health or barrier properties of the skin), pH balancing, lubricating (e.g. to reduce chapping of the skin by rubbing of clothing upon the skin), relieving (pain and irritation), and/or cleaning the skin. Examples of specific classes of medicaments include, but are not limited to, the following: astringents, antiseptic agents, antioxidants, antimicrobial agents, antifungal agents, deodorants, enzyme inhibitors, emollients, skin protectants, and so forth. These and other medicaments or skin health benefit agents are known in the art.

In order that the medicaments applied to the body-side surface stay upon the exposed portion of the substrate or porous fabric and do not substantially migrate into or through the topsheet. The medicaments will typically be provided in the form of a composition including one or more skin health benefit agents as well as additional solvents, surfactants, stabilizers, viscosity/rheology modifiers, suspending agents and/or other agents to achieve the desired physical properties. The medicament compositions desirably comprise high viscosity liquids or emulsions, gels, semi-solids or solids at room temperature and which are capable of being extruded as a liquid. In this regard, the medicament compositions should have a melting point of at least about 25° C. and desirably has a melting point between about 30° C. and about 100° C., and still more desirably has a melting point between about 40° C. and about 80° C. In addition, the medicament compositions are desirably capable of adequately adhering to the topsheet such that the medicament does not substantially migrate into or through the topsheet. However, the medicament compositions are desirably not so solid nor do they adhere so strongly to the topsheet that the medicament is prevented from being transferred from the topsheet to the skin when the article is worn. In this regard, the medicament compositions desirably have a low shear viscosity of from about 50 to about 1,000,000 centipose and more desirably between about 50,000 and about 800,000 centipose at a temperature of about 60° C. In addition, the penetration hardness of the medicament composition desirably ranges between about 5 and about 350 millimeters and still more desirably between about 5 and about 150 millimeters.

As an example, the body-side portion of a liner or topsheet can be provided having a hydrophilic lotion formulation on the surface thereof wherein the hydrophilic lotion formulation comprises about 10 to about 90 weight percent of a hydrophilic solvent, from about 5 to about 90 weight percent of a polyethylene glycol having a molecular weight of at least about 720 and from about 20 to about 60 weight percent of a fatty alcohol. The hydrophilic solvent component desirably comprises one or more propylene glycols and/or low molecular weight polyethylene glycols. The fatty alcohol desirably comprises an alcohol having a carbon chain length of from about C₁₄-C₃₀ including, but not limited to, cetyl alcohol, stearyl alcohol, arachidyl alcohol, behenyl alcohol and mixtures thereof. Hydrophilic lotion formulations of this nature are described in more detail in commonly assigned U.S. patent application Ser. No. 09/298,313 (and related case WO 00/64500, published on Nov. 2, 2000); the entire contents of the aforesaid application is incorporated herein by reference.

As a further specific example, a body side liner can be provided having a lotion formulation on the outer surface thereof wherein the lotion formulation comprises about 5 to about 95 weight percent of an emollient, about 5 to about 95 weight percent of a solidifying agent and, 0 to about 25 weight percent of a viscosity enhancer. Suitable emollients include, but are not limited to, petrolatum based oils, vegetable based oils, animal based oils, mineral oils, silicones, synthetic oils, lanolin and its derivatives, esters, branched esters, gurbet esters, glycerol esters and derivatives, propylene glycol and its derivatives, alkoxylated carboxylic acids, alkoxylated alcohols, fatty alcohols, triglycerides, alkyl hydroxystearates and mixtures thereof. Exemplary solidifying agents (primary function to solidify the composition so that the composition is a solid at room temperature) include, but are not limited to, C₁₆ or greater alkyl silicones, fatty acid esters with a melting point of about 35° C. or greater, C₁₆ or greater alkyl hydroxystearates, alkoxylated alcohols, alkoxylated carboxylic alcohols, hydrogenated animal and vegetable oils and waxes or modified waxes like bayberry wax, beeswax, carnuba wax, ceresin, lanolin wax, paraffin, rice bran wax, synthetic spermaceti wax, microcrystalline wax, shellac wax, montan wax, fluoranated waxes and mixtures and combinations thereof. Suitable viscosity/rheology modifiers include, but are not limited to, polyolefin resins and polymers, ethylene/vinyl acetate copolymers, silica, treated silica, talc, clays and organically modified clays, colloidal silicon dioxide, zinc stearate, cetyl hydroxy ethyl cellulose, and combinations and mixtures thereof. Such lotion formulations are described in more detail in commonly assigned U.S. Pat. Nos. 6,149,934 and 6,217,890 and U.S. patent application Ser. No. 09/671,357; the entire contents of the aforesaid patents and applications are incorporated herein by reference.

As further examples, the medicament composition can comprise a high viscosity oil-in-water emulsion comprising a skin health benefit agent, a surfactant and water. Exemplary oil-in-water medicaments are also described in U.S. patent application Ser. No. 09/596,162, the entire contents of which are incorporated herein by reference. While various medicaments and/or medicament compositions are disclosed herein it will be appreciated that other medicaments and/or medicament compositions can be used in conjunction with the present invention. Additional medicament compositions include, but are not limited to, those described in U.S. patent application Ser. Nos. 09/382,018 (and related case WO 00/64501, published on Nov. 2, 2000) and 09/379,929 (and related case WO 00/64409, published on Jun. 20, 2001); the entire contents of each of which are incorporated herein by reference.

Particularly useful exemplary chemistries, which are suitable for use in the present invention include, natural lipid materials like but are not limited to: Essential and/or nonessential fatty acids, sterols and derivatives,

An example of a lipid composition that was successfully demonstrated contained the following ingredients: phospholipid SV, a biomimetic cosmetic emollient composed of diester and triester phophatides (described as stearamidopropyl phospathatidyl PG-dimonium chloride and cetyl alcohol); phospholipid CDM, a biomimetic cosmetic emollient composed of diester and triester phophatides; Monamid 150-IS, an isostearic diethanolamide; propylene glycol; squalene, an open-chain isoprenoid hydrocarbon, C₃₀; and octyl stearate. Other examples of the lipid composition included essential and/or nonessential fatty acids, sterols and derivatives.

### Application to Topsheet

The medicament and/or medicament composition may be applied to the substrate in a manner to allow the release of the medicaments from the substrate onto the wearer's skin through normal contact, wearer motion and/or body heat. Thus, the medicament and/or medicament composition is desirably applied to the body-side surface of the topsheet such that the majority of the medicament remains at or immediately adjacent the surface of the body-side surface of the topsheet. As noted above, this can be accomplished through the use of additives, such as surfactants, stabilizers, viscosity modifiers and/or other agents, to the medicaments. The addition of fillers to the medicament or medicament composition is also contemplated. Still more desirably, the medicament and/or medicament composition is applied in a manner to form a portion extending above the fabric having a substantially rounded or substantially semi-circular cross-sectional shape. Further, the amount of medicament and/or medicament composition applied to the body-side surface of the topsheet is determined by considering the percent of the medicament normally released during use as well the amount of medicament necessary to provide a benefit to skin health. It is further contemplated that the use of the surfactants or other additives mentioned above may also enhance the transferability of the medicament or medicament composition. Furthermore, it is also contemplated that a boundary layer or the like may be included, such as is disclosed in co-pending, commonly assigned U.S. patent application Ser. No. 09/938347, entitled “Treated Substrate With Improved Transfer Efficiency Of Topical Application” and filed on Aug. 24, 2001. While the effective amount for any given medicament or medicament composition may vary, typically it will be desirable to apply the medicament or medicament composition to the body-side surface of the topsheet in an amount between about 0.05 mg/cm² and about 50 mg/cm² and still more desirably between about 1 mg/cm² and about 25 mg/cm².

In addition, in many embodiments of the present invention it is desirable that the amount of medicament and/or medicament composition upon the body-side surface of the topsheet is provided such that it is uniformly present across the applied area, i.e. the treated regions; however, in some embodiments, due to the medicament pattern selected, uniformity of application across the article as well as in density of application, may not be desired. Desirably, in the treated area, the amount of medicinal composition per square centimeter varies by less than about 5% from the average amount of medicinal composition per square centimeter and still more desirably varies by less than about 2% from the average amount of medicinal composition per square centimeter. The medicament or medicament composition is desirably applied to the body-contacting regions of the substrate and more desirably upon the body contacting region of the topsheet. The medicament or medicament composition can be applied across the entire body-contacting regions of the topsheet or across limited or selected regions. Desirably, the medicament and/or medicament composition is applied in lines extending substantially in the machine direction. However, the medicament and/or medicament compositions can be applied in any of numerous patterns and densities and including aesthetically pleasing patterns and images. The pattern or patterns may be repeating or non-repeating and may be randomly placed. The present invention also contemplates the uniform coating or application of the medicament and/or medicament compositions.

The medicament or medicament composition is desirably applied to the substrate in a desired pattern upon the topsheet wherein at least one of the dimensions of the pattern, in terms of length (L) and width (W), is less than about 4 millimeters, and more desirably, in some embodiments, is less than about 1 millimeter. The patterns desirably comprise a series of continuous and/or substantially continuous lines. The lines can comprise parallel lines and/or intersecting lines and further the lines can be straight, zigzag, sinusoidal or curved. Further, the lines can be provided upon the topsheet in directions parallel, perpendicular or angled to the machine direction of the topsheet. Further, the medicaments or medicament compositions can be applied in a controlled random manner wherein the amount of medicament per unit area is controlled but the specific orientation and direction of the medicament applied thereto experiences some degree of variation and is not a repeating pattern. The patterned areas can themselves comprise a dot matrix pattern or closely spaced and/or overlapping lines which are not visible to the naked eye. Desirably the medicament is applied to the substrate in a series of continuous or substantially continuous lines having a width less than about 4 mm. In one or more embodiments it is desirable lines have a width less than about 1 mm and still more desirably a width less than about 0.9 mm and even more desirably a width between about 0.9 mm and about 0.05 mm. In an alternative embodiment it is desirable for the lines to have a width in the range of about 1.5 mm to about 3 mm and more desirably in the range of about 1.5 to about 2.5 mm. The individual lines can be spaced as desired and desirably have a spacing less than about 2.5 cm and still more desirably about 1 cm or less.

The medicament can be applied to the body-side surface of the topsheet using various forms of equipment capable of a precise, uniform application of a liquid in a desired pattern. Such equipment includes, but is not limited to, equipment commonly used for the registered application of adhesives as well as print heads and printing devices such as, for example, continuous ink-jet (CIJ) printers, drop-on-demand (DOD) printers, electrostatic printers and so forth. Specific equipment suitable for use in the present invention includes, by way of example only, MELTEX EP11 coating die, CONTROL WEAVE (CW-200) Applicators and the Hot Melt Rotary Screen Coating System all from Nordson Corporation of Norcross, Ga. With regard to the CW-200 applicators from Nordson Corporation, in order to achieve the precise application required for the present invention, it is desirable to run the applicator with the draw air off. As a further example, suitable applicators also include PHASER 5 print heads available from Tektronix, Inc. (having offices in Beaverton, Oreg.). With regard to the print heads it is possible to run the print head in a continuous process. Other suitable applicators include a piezoelectric jet (hereinafter “piezo jet”), available from Spectra, Inc. (Lebanon, N.H.) or a micro-dispense valve (hereinafter “valve jet”), desirably a solenoid valve jet, such as the one described in the commonly assigned, copending U.S. patent applications Ser. Nos. ______/______ and ______/______ and respectively entitled “Apparatus To Produce Topography, Unique Fluid Handling Properties And Bonding Properties On And Within Substrates, And A Method For Producing The Same” and “Material Having One Or More Applications Which Produce Topography, Unique Fluid Handling Properties And/Or Bonding Properties Thereon Or Therein”, both filed on Nov.______, 2001.

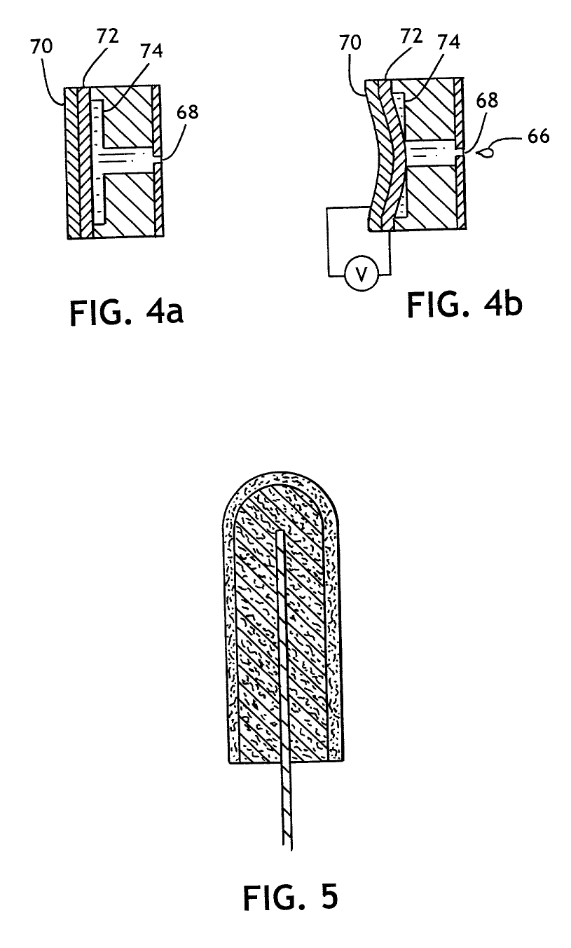

In general the process of creating discrete segments via a piezo jet may be described as follows. An asynchronous droplet stream may be generated by a rapid volume change in a fluid chamber 74 connected to an orifice 68 as illustrated in FIGS. 4a and 4b. The fluid static pressure at the orifice 68 is maintained at a value less than that needed to overcome surface tension forces and in some cases below atmospheric pressure. A rapid potential pulse applied to the piezoelectric element 70 causes a displacement of the element 70 and a metal support adjacent to the piezoelectric material 70. The rapid displacement reduces the chamber 74 volume, expelling a droplet 66 from the orifice 68. The displacement must provide sufficient kinetic energy to accelerate a portion of the fluid beyond the escape velocity of the orifice 68. Once expelled from the orifice 68, the droplet 66 desirably contacts a substrate (not shown), such as that described in the present invention, whereby it forms a discrete segment thereon.

If the medicament is applied via piezo jet, it is desired that the process viscosity of the medicament applied to the substrate be up to about 25 centipoise, more desirably about 4 centipoise to about 20 centipoise, and most desirably about 8 centipoise to about 14 centipoise, at the time of application. Furthermore, it is desired that the pattern of medicament include a matrix of discrete segments having a volume of between about 3 and about 200 picoliters. Further still, it is desired that the discrete segments may be placed upon substrate in a frequency between about 10 kHz to about 50 kHz, and more desirably applied to the substrate at a frequency of about at a frequency of up to about 40 kHz. As above, it is of note that the frequency expressed in Hz is not the number of times the applicator must fire to produce one segment, but is the number of times the applicator may fire in a given time period so as to produce a number of discrete segments over the desired region or regions of the substrate.

If the medicament is applied via valve jet, it is desired that the viscosity of the medicament applied to the substrate in a range of about 1 centipoise to about 300 centipoise, more desirably about 4 centipoise to about 50 centipoise at the time of application. Furthermore, it is desired that the pattern of medicament include a matrix of discrete segments having a volume of between about 5 and about 400 nanoliters. Further still, it is desired that the discrete segments may be placed upon substrate at a frequency between about 1 Hz to about 2 kHz, and more desirably at a frequency of about 500 Hz to about 1.2 kHz.

It is of note that some of the frequencies which are identified as desirable may not currently be obtainable with one or more of the applicators discussed herein; however, as the application frequency is currently limited by mechanical limitations (e.g. undesired electrical communication or mechanical vibration as a result of the proximity of the electronics or orifices to one another) and because it is expected that improvements in the technology will continue to be made so as to allow higher frequency operation with the applicators, it is to be understood by one skilled in the art that such higher frequency operation are contemplated by and intended to be included in the present invention and would be desirable. Stated another way, some of the application methods are currently limited by mechanical limitations and if these mechanical limitations are overcome, the substrates claimed and described herein would be capable of production therewith. Furthermore, as it would be desirable to operate one or more of the applicators discussed herein at higher frequency, the use of an applicator capable of higher frequency application is contemplated by and intended to be included in the process of the present invention.

As indicated above, the patterned regions can themselves comprise a matrix or assemblage of individual dots or lines. Desirably, the patterned regions comprise medicament and/or medicament compositions applied in a matrix of discrete dots or segments. Such a matrix or pattern is believed to lessen the loss of liquid handling properties in the treated areas. In this regard, the matrix can have a dot resolution of any quantity so long as it does not substantially interfere with the desired characteristics of the article in question. Further, each of the drops or segments desirably contains substantially the same amount of medicament and/or medicament composition. The individual dots or segments desirably comprise from about 3 picoliters to about 400 nanoliters of the medicament or medicament composition. Still further, the medicaments and/or medicament compositions are desirably applied to the topsheet such that the dots or segments have a cross-section having a substantially round or substantially hemispherical shape extending above the body-side surface of the topsheet. Although segments having shapes other than those of a substantially round or substantially hemispherical shape are contemplated by the present invention, it has been found that the substantially round or substantially hemispherical shape is more efficacious with regard to achieving the desired transfer of medicament or medicament compositions and therefore decreases the amount of medicament or medicament composition which needs to be applied to the substrate to achieve the desired results.

In reference to FIG. 2, a cross-sectional representation of a topsheet 40 is provided wherein the body-side surface 42 of the topsheet 40 contains medicament composition 44 having a hemispherical shape extending there above. By providing dots and/or segments having a rounded shape a larger amount of the medicament and/or medicament composition is provided per surface area of the topsheet relative to medicaments applied and having a flat structure. By utilizing small drops and/or segments the melted or liquid medicament and/or medicament composition cools and/or solidifies quicker thereby allowing formation of the round shape above the body-side surface of the topsheet. While not wishing to be bound by theory, the small size is further believed to facilitate transfer of the entire segment and/or dot. In addition, by utilizing numerous discrete dots or segments within the treated area the area of exposed edges is increased. In this regard, the increased edge area is believed to facilitate removal of the treatment from the sheet to the wearer, particularly when transfer is facilitated by mechanical action, i.e. movement of the body against the topsheet.

Specifically, the present invention is directed to a process for producing a medicament treated material, the process including providing a substrate; and applying a medicament composition to the substrate in discrete segments; wherein the medicament composition is present on the substrate in a frequency of about 1 Hz to about 100 kHz. The step of providing a substrate may include the provision of a woven, nonwoven, film or laminate thereof. The step of applying the medicament or medicament composition to the substrate may comprise the projection of the medicament from a discharge orifice. It is also contemplated that the discrete segments may be located upon selected regions of the substrate. The process of the present invention may also include the application of the medicament or medicament composition to a substrate in a pattern, which may be repeating or non-repeating. As discussed in more detail herein, it is contemplated that the step of applying the medicament or medicament composition to the substrate may be achieved by any non-contacting application means or applicator means, but is desirably achieved by an ink-jet application, such as a valve jet or piezo jet, or a similar application means.

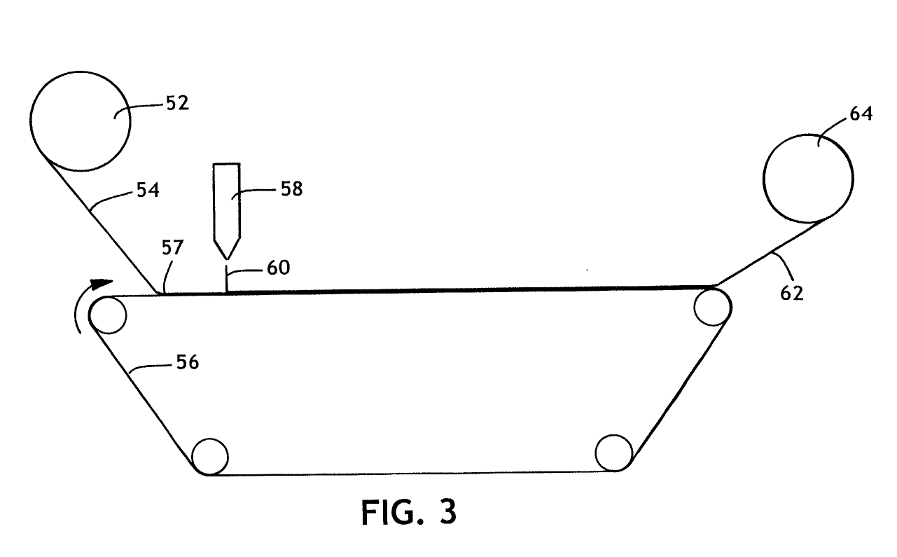

An exemplary process for forming the topsheets of the present invention is depicted in FIG. 3. Substrate 54, such as a porous fabric or a nonwoven web, is unwound from roll 52 and fed onto a moving belt or screen 56. The belt 56, with substrate 54 thereover, travels under applicator 58 and medicament composition 60 is applied to the upper surface 57 of substrate 54. The medicament composition 60 is then cooled or dried, if needed, and in this regard most often the medicament composition will cool and resolidify quickly after exposure to ambient air. The treated substrate 62 may subsequently be wound on a winder roll (not shown) forming roll 64 of treated fabric. In the alternative, the treated substrate can be converted immediately without first being wound and stored in that form. Alternatively, the topsheet can be treated in the finished article. A personal care article, such as a diaper, can be fabricated and travel along a belt in an uncontracted state and with the topsheet facing-up. Application of the medicament composition upon the exposed topsheet can be applied via the equipment described herein above using registered treatment techniques as are known in the art, as well as those discussed above. While a particular process of fabricating a treated substrate is disclosed herein, it will be appreciated to those skilled in the art that various modifications and/or changes can be made without departing from the spirit of the present invention. As an example, the substrate can be made in-line instead of having been previously made and wound in roll form. Still further, the body-side layer of a topsheet can be treated and medicament composition applied thereto in the converted form using registered application methods.

In addition, to facilitate removal of the medicament and/or medicament treatments from the substrate, a surfactant can be applied to the fabric prior to deposition of the medicament or medicament composition. The surfactant can be used to modify the affinity of the medicament or medicament composition for the porous material comprising the topsheet. In this regard, the medicament or medicament composition desirably has sufficient affinity for the porous material such that the treatment stays upon the topsheet, but not such a high affinity that it does not transfer to the skin. Desirably, the medicament or medicament composition has a higher affinity for the skin than the matter comprising the porous material. A surfactant believed suitable for use with the present invention includes CETIOL, available from the Henkel Corporation, and comprises an ethoxylated ester derivative of myristic acid. Additional surfactants believed suitable with the present invention include, but are not limited to, silicone and polyethylene glycol based surfactants. The surfactant can be uniformly treated across the body contacting regions of the topsheet or, in the alternative, can be applied so as to overlap only those regions that will contain the medicinal treatments.

While various patents and other reference materials have been incorporated herein by reference, to the extent there is any inconsistency between incorporated material and that of the written specification, the written specification shall control. In addition, while the invention has been described in detail with respect to specific embodiments thereof, it will be apparent to those skilled in the art that various alterations, modifications and other changes may be made to the invention without departing from the spirit and scope of the present invention. It is therefore intended that the claims cover or encompass all such modifications, alterations and/or changes.

We claim:

1. An article, comprising:
a topsheet having a body-facing surface, said topsheet having medicinal composition applied in a pattern to a region of the body-facing surface of the topsheet in an amount between about 0.05 mg/cm² and about 50 mg/cm² and wherein said pattern comprises individual segments having at least one dimension of about 4 millimeters or less; and
an absorbent material positioned adjacent said topsheet.

2. The article of claim 1, wherein the article further comprises a backsheet; and wherein the absorbent material positioned between said topsheet and said backsheet.

3. The article of claim 2, wherein the backsheet is a liquid-impervious backsheet.

4. The article of claim 1, wherein said pattern comprises a plurality of substantially continuous lines having a width less than about 4 millimeters.

5. The article of claim 4, wherein said pattern of substantially continuous lines have a width less than about 1 millimeters.

6. The article of claim 1, wherein said medicinal composition is present upon said body-facing surface of said topsheet in an amount between about 1 and about 25 mg/cm².

7. The article of claim 1, wherein each square centimeter of said region containing said patterned medicinal composition has a substantially uniform pattern application of medicinal composition and wherein the amount of medicinal composition varies in said region by less than about 5% from the average amount of medicinal composition per square centimeter.

8. The article of claim 7, wherein each square centimeter of said region containing said patterned medicinal composition has a substantially uniform pattern application of medicinal composition and wherein the amount of medicinal composition varies in said region by less than about 2% from the average amount of medicinal composition per square centimeter.

9. The article of claim 1, wherein said pattern comprises a matrix of discrete segments and further wherein the discrete segments have a length and width less than about 0.9 mm.

10. The article of claim 9, wherein said discrete segments are positioned upon said topsheet in a frequency between about 1 Hz and about 100 kHz.

11. The article of claim 9, wherein said discrete segments have a volume of between about 3 picoliters and about 400 nanoliters.

12. The article of claim 1, wherein the medicament further includes a filler.

13. The article of claim 1, wherein the topsheet comprises a woven, nonwoven, or laminate thereof.

14. An absorbent article, comprising:
a topsheet having a body-facing surface, said topsheet comprising a porous material and having medicinal composition applied in a pattern upon the body-facing surface of the topsheet in an effective amount and wherein said pattern comprises a matrix of discrete segments having a volume of between about 3 picoliters and about 400 nanoliters; and
an absorbent material positioned adjacent said topsheet.

15. The absorbent article of claim 14, wherein the article further comprises a backsheet; and
wherein the absorbent material positioned between said topsheet and said backsheet.

16. The absorbent article of claim 14, wherein said discrete segments have a substantially semicircular cross-section extending above the body-facing surface of the topsheet.

17. The absorbent article of claim 14, wherein said discrete segments are positioned upon said topsheet in a frequency between about 1 Hz and about 100 kHz.

18. The absorbent article of claim 14, wherein said discrete segments are located only upon selected regions of the topsheet.

19. The absorbent article of claim 14, wherein said discrete segments are located only within spaced lines extending across the topsheet and wherein said lines have a width less than about 4 mm.

20. The absorbent article of claim 14, wherein the medicinal composition further includes a filler.

21. The absorbent article of claim 14, wherein the absorbent article comprises one or more components of a personal care product.

22. The absorbent article of claim 21, wherein the personal care product is selected from a diaper, training pant, absorbent underpants adult incontinence product, sanitary wipe, wet wipe, feminine hygiene product, wound dressing, nursing pad, time release patch, bandage, mortuary product, veterinary product, hygiene and absorbent product.

23. A process for producing a medicament treated material, said process comprising: providing a substrate; and
applying a medicament composition to the substrate in discrete segments;
wherein the medicament composition is present on the substrate in a frequency of about 1 Hz to about 100 kHz.

24. The process of claim 23, wherein the step of applying the medicament or medicament composition to the substrate comprises the projection of the medicament from a discharge orifice.

25. The process of claim 23, wherein the step of providing a substrate comprises providing a woven, nonwoven, film or laminate thereof.

26. The process of claim 23, wherein the step of providing a substrate comprises providing a web of fibers, wherein said web of fibers comprises a nonwoven material.

27. The process of claim 23, wherein said discrete segments are located only upon selected regions of the substrate.

28. The process of claim 23, wherein the medicament contains a lipid.

29. The process of claim 23, wherein the medicament is applied to the substrate at a temperature of about 15° C. to about 135° C.

30. The process of claim 23, wherein the discrete placement is achieved by valve jet, piezo jet, spraying or any non-contact means of application.

31. The process of claim 30, wherein the discrete placement is achieved by piezo jet.

32. The process of claim 31, wherein the medicament applied to the substrate has a viscosity of up to about 25 centipoise at the time of application.

33. The process of claim 31, wherein the medicament applied to the substrate has a viscosity of about 4 centipoise to about 20 centipoise at the time of application.

34. The process of claim 31, wherein the medicament applied to the substrate has a viscosity of about 8 centipoise to about 14 centipoise at the time of application.

35. The process of claim 31, wherein said pattern comprises a matrix of discrete segments having a volume of between about 3 and about 200 picoliters.

36. The process of claim 31, wherein said discrete segments are placed upon substrate in a frequency between about 10 and about 100 drops per cm².

37. The process of claim 23, wherein the medicament is applied to the substrate at a frequency of about 10 kHz to about 50 kHz.

38. The process of claim 36, wherein the discrete placement is achieved by valve jet.

39. The process of claim 38, wherein the medicament applied to the substrate has a viscosity of about 1 centipoise to about 300 centipoise at the time of application.

40. The process of claim 38, wherein the medicament applied to the substrate has a viscosity of about 4 centipoise to about 50 centipoise at the time of application.

41. The process of claim 38, wherein said pattern comprises a matrix of discrete segments having a volume of between about 5 and about 400 nanoliters.

42. The process of claim 23, wherein the medicament is applied to the substrate at a frequency of about 1 Hz to about 2 kHz.

43. The process of claim 42, wherein the medicament is applied to the substrate at a frequency of about 500 Hz to about 1.2 kHz.
